(19) United States
(12) Patent Application Publication
(10) Pub. No.: US 2015/0202282 A1
(43) Pub. Date: Jul. 23, 2015

(54) PCV2 ORF2 VIRUS LIKE PARTICLE WITH FOREIGN AMINO ACID INSERTION

(71) Applicant: Boehringer Ingelheim Vetmedica, Inc., Saint Joseph, MO (US)

(72) Inventors: Eric Vaughn, Ames, IA (US); Merrill Schaeffer, Saint Joseph, MO (US)

(21) Appl. No.: 14/592,562

(22) Filed: Jan. 8, 2015

Related U.S. Application Data
(63) Continuation of application No. 12/812,590, filed on Jul. 12, 2010, now abandoned.
(60) Provisional application No. 61/017,863, filed on Dec. 31, 2007.

(51) Int. Cl. A61K 39/145; C07K 14/00; C07K 14/005; C12N 7/00; A61K 39/012

(52) U.S. Cl. CPC A61K 39/145; C12N 7/00; A61K 39/012; C07K 14/005; C07K 14/00; C12N 2750/10034; C12N 2750/10022; A61K 2039/5258

(57) ABSTRACT

The present invention comprises methods and compositions related to the production and use of amino acid sequences. In particular, PCV2 ORF2 is shown to be useful as a virus-like particle which produces amino acid sequences that retain their immunogenicity or antigenicity when the DNA encoding the PCV2 ORF2 is inserted into an expression system. DNA sequences that are foreign to PCV2 can be attached “in-frame” to the ORF2 DNA and the entire sequence, including the DNA foreign to PCV2, is expressed. It was shown that such sequences retain their antigenicity and therefore their potential utility in immunogenic compositions.

### SEQUENCE LISTING

This application contains a sequence listing in computer readable format, the teachings and content of which are hereby incorporated by reference.

### BACKGROUND OF THE INVENTION

1. Field of the Invention

The present application is concerned with the use of a porcine circovirus type 2 (PCV2) virus-like particle as a vector for expressing desired amino acid sequences. More particularly, the present application is concerned with the use of such a vector for expressing immunogenic amino acid sequences of pathogens and the subsequent use of such expressed immunogenic amino acid sequences in immunogenic compositions. Still more particularly, the present application is concerned with the use of open reading frame 2 (ORF2) from PCV2 as a vector for expressing desired amino acid sequences. Even more particularly, the present application is concerned with the insertion of foreign amino acid sequences into PCV2 ORF2 and the subsequent expression of the ORF2 sequence and the foreign amino acid sequence in an expression system. Still more particularly, the present application is concerned with the use of such expressed sequences in immunogenic compositions.

2. History of the Prior Art

The Porcine Circovirus 2 (PCV2) open-reading ORF2 gene can be expressed in insect cell culture. It has also been shown that the PCV2 ORF2 protein likely assembles into virus-like particles (VLP). These VLP are essentially empty PCV2 capsids and are highly immunogenic. The very first description of fusing a relevant peptide region to a virus-like particle may been in 1986 (Delpeyroux et al. 1986. A poliovirus neutralization epitope expressed on hybrid hepatitis B surface antigen particles. Science. July 25; 233(4762):472-5 (the teachings and content of which are hereby incorporated by reference)).

It has been shown that a monoclonal antibody (3E2) directed against the CSL 30-mer has been shown to provide some efficacy against Cryptosporidium infection in mice via passive immunotherapy. The Circumsporozoite ligand (CSL) is an immunogenic protein sequence of 30 amino acids (30-mer). It has been determined that the monoclonal antibody 3E2 recognizes an epitope within the 30 amino acids of the N-terminus from CSL. The protein sequence of the 30-mer is AINGGGATLPQKLYLTPNVLTAGFAPYIGV (SEQ ID NO. 1). The peptide for this CSL 30-mer can be generated by chemical synthesis, and then used in a vaccine preparation to induce an antibody response in a vaccinated animal. It is possible to generate an anti-CSL 30-mer immune response by immunization with chemically synthesized CSL peptide that has been combined with adjuvant. However, the costs of using chemically synthesized CSL peptide in a commercial vaccine are prohibitive.

Influenza viruses are divided into three types, designated A, B and C. Influenza types A and B are responsible for epidemics of respiratory illness that occur almost every winter and are often associated with increased rates for hospitalization and death. Influenza type A viruses are divided into subtypes based on differences in two viral proteins called hemagglutinin (HA) and neuraminidase (NA). The influenza virus matrix 1, otherwise known as M1, is a critical protein required for assembly and budding. HA and NA interact with influenza virus M1; HA associates with M1 via its cytoplasmic tail and transmembrane domain. The M2 protein is critical in the replication cycle of influenza viruses and is also an essential component of the viral envelope because of its ability to form a highly selective, pH-regulated, proton-conducting channel. The M2 channel allows protons to enter the virus' interior, and acidification weakens the interaction of the M1 protein with the ribonuclear core.

The influenza M2-protein is a tetrameric, type III transmembrane protein that is abundant on virus-infected cells. The M2e is the external domain of the influenza A M2-protein. The human influenza A M2e-sequence is only 23 to 24 amino acids long, and has remained nearly unchanged throughout the occurrence of numerous epidemics and two major pandemics. Of note, although many swine influenza A strains have emerged in recent years, the M2e region of swine Influenza A viruses has also remained relatively unchanged. Because of the conserved nature of the M2e region target sequence in Influenza A virus strains, the M2e is considered to be “universal” antigen for influenza vaccines. One preferred amino acid sequence of the 24-mer M2e region target sequence is MSLLTEVETPIRNEWGCRCNDSSD (SEQ ID NO. 6) (also referred to herein as M2ae1). The peptide for this influenza A M2e region 24-mer can be generated by chemical synthesis, and then used in a vaccine preparation to induce an antibody response in a vaccinated animal. It is possible to generate an anti-influenza A M2e 24-mer immune response by immunization with chemically synthesized M2e peptide that has been combined with adjuvant. However, the costs of using chemically synthesized M2e peptide in a commercial vaccine are prohibitive.

It has not been suggested to use the virus-like particle properties of PCV2 ORF2 as a system or machinery to express amino acid sequences or proteins, much less amino acid sequences or proteins unrelated or foreign to PCV2 ORF2. Accordingly, it has also not been suggested to use the virus-like particle properties of PCV2 ORF2 as a vector for producing an immunologically relevant peptide, including the representative examples of a 30-mer CSL peptide or a 24-mer influenza A M2e peptide, and subsequently using such peptides in an immunogenic composition or vaccine.

### SUMMARY OF THE INVENTION

The present invention demonstrates that it is possible to use the PCV2 ORF2 as a virus-like particle that includes segments therein or attached thereto that are foreign to native PCV2 ORF2, but that still retain their immunogenicity or antigenicity. Specifically, examples demonstrating such use are provided herein. In preferred forms, nucleic acid sequence segments foreign to PCV2 ORF2 can be attached to or integrated in the ORF2 sequence and expressed in an expression system. Advantageously, the expressed amino acid segments retain their immunological properties or antigenicity. In preferred forms, both the PCV2 ORF2 and the foreign amino acid sequence retain their immunological properties. The foreign sequence can be attached to or integrated with the PCV2 ORF2 sequence at the amino or carboxyl terminus or end, or any position therebetween. The expressed foreign amino acid sequences are preferably of a length of at least 8, and more preferably between 8 and 200 amino acids in length, thereby making the inserted foreign nucleic acid segments at least 24 nucleotides, and more preferably between 24 and 600 nucleotides in length. The preferred length for any specific nucleic or amino acid segment will be determinable by those of skill in the art, but will preferably be selected based on the immunological response the amino acid segment induces in an animal after administration thereof. Preferred foreign amino acid segments will reduce the incidence of or lessen the severity of clinical and/or pathological or histopathological signs of infection by a pathogen against which the segment induces an immune response. Preferably, the segment will have at least 80%, more preferably 85%, still more preferably 90%, even more preferably 92%, 93%, 94%, 95%, 96%, 97%, 98%, and most preferably at least 99% sequence homology with an amino acid segment known to induce an immunological response in an animal. Even more preferably, the segment will have at least 80%, more preferably 85%, still more preferably 90%, even more preferably 92%, 93%, 94%, 95%, 96%, 97%, 98%, and most preferably at least 99% sequence identity with an amino acid segment known to induce an immunological response in an animal. Preferably, the amino acid segment will induce an immune response that reduces the incidence of or lessens the severity of clinical, pathological, or histopathological signs of infection from a pathogen from which the amino acid segment is derived when the amino acid segment is administered to an animal in need thereof. Thus, one aspect of the present invention identifies amino acid segments or the nucleic acid sequences expressing amino acid segments that reduce the incidence of or lessen the severity of clinical, pathological, and/or histopathological signs of infection by a specific pathogen. These amino acid segments can also be used to deduce the nucleic acid sequence expressing the amino acid segment, which is then inserted into a vector, preferably PCV2 ORF2, expressed in an expression system, preferably a baculovirus expression system, recovered, and finally administered to an animal in need thereof. In some preferred forms, the expressed product is left intact and the foreign amino acid segment is not separated from the expressed sequence, and in other preferred forms, the foreign amino acid segment is removed or excised from the expressed sequence. Thus, in the case of the CSL sequence described below, the foreign CSL sequence can be left as a part of the ORF2 sequence after the expression thereof and administered to an animal in need thereof as a chimeric sequence, or the foreign CSL sequence can be excised from the ORF2 sequence and administered separately or simultaneously to an animal in need thereof.

In one preferred embodiment, the present invention provides a specific application using the CSL 30-mer as an example. The CSL 30-mer is provided herein in a cost-effective, immunologically relevant manner by the fusing the CSL 30-mer to the carrier protein, PCV2 ORF2. This is done by creating a PCV2 ORF2 CSL Baculovirus in a manner such that the expression thereof results in the CSL 30-mer being attached in frame as a “tail” on the carboxyl or amino end of the PCV2 ORF2 sequence, or is integrated in-frame within the PCV2 ORF2 sequence. The example herein attaches the CSL 30-mer tail at the carboxyl end of the PCV2 ORF2 protein, but those of skill in the art will understand that this location can be adjusted as desired, as further evidenced by the examples of the swine influenza amino acid segment which was attached at both the amino end of the PCV2 ORF2 as well as within the ORF2 sequence. Thus, when insect cells are infected with the PCV2 ORF2 CSL Baculovirus, there will be generation of a chimeric PCV2 ORF2 VLP that also contains the CSL 30-mer as a “tail”. This PCV2 ORF2 can serve as a carrier for the CSL 30-mer.

The present invention also demonstrates that fusing the CSL 30-mer to PCV2 ORF2 is immunologically relevant and has been reduced to practice. The immunological relevance of chimeric PCV2 ORF2 CSL expression in insect cells was detected by antibodies directed towards the PCV2 ORF2 protein and also by antibodies directed towards the CSL 30-mer.

Previous work has demonstrated that PCV2 ORF2 protein can be expressed in insect cell culture to very high levels with a minimal amount of downstream processing. This application demonstrates that the CSL 30-mer can be fused as an in-frame “tail” on the carboxyl end of the PCV2 ORF2 protein so that the PCV2 ORF2 capsid will serve as a carrier for the CSL 30-mer. The chimeric PCV2 ORF2 CSL protein is also expressed to high levels in insect cell cultures with a minimal amount of downstream processing, which in turn can be used as antigen in cost-effective vaccine preparations. Advantageously, although the monoclonal antibody 3E2 directed against the CSL 30-mer has been shown to provide some efficacy against Cryptosporidium infection in mice via passive immunotherapy, it is likely that a polyclonal antibody response directed towards the CSL 30-mer may induce a more robust and efficacious response against Cryptosporidium infection.

Some potential uses for the chimeric PCV2 ORF2 CSL antigen include individual vaccination, passive immunization, and serum therapy. For individual vaccination, chimeric PCV2 ORF2 CSL antigen is administered to animal in need thereof in order to vaccinate individual animals for the induction of a protective humoral and/or cell-mediated response against Cryptosporidium infection. For passive immunization, chimeric PCV2 ORF2 CSL antigen is administered for the induction of a robust humoral and/or cell-mediated response directed towards the CSL 30-mer that can be passively passed on to nursing offspring. This passive maternal immunity will in turn reduce or prevent Cryptosporidium infection in the offspring. For serum therapy, Administration of chimeric PCV2 ORF2 CSL antigen for the induction of a robust humoral response directed towards the CSL 30-mer that can be used in serum therapy. Large animals (i.e. horses) can be hyperimmunized with chimeric PCV2 ORF2 CSL for generation of anti-CSL 30-mer antisera. The antisera can be administered orally to clinically affected animals to reduce clinical disease caused by Cryptosporidium infection.

Based on the present invention, those of skill in the art will also recognize that amino acid segments such as the CSL 30-mer and swine influenza 24-mer could be fused with another virus-like particle carrier (i.e. PCV1, parvoviruses, enteroviruses, and other viruses with capsid structure). Additionally, the PCV2 ORF2 VLP could be used to package and carry foreign DNA (i.e. a DNA vaccine encoding for a relevant antigen or for in use in gene therapy).

One further aspect of the present invention is the use of amino acids expressed using the methods of the present invention in antigenic or immunogenic compositions or vaccines. Such compositions or vaccines could be further combined with adjuvants, pharmaceutical acceptable carriers, protectants, and/or stabilizing agents.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol (BF Goodrich, Ohio, USA) are particularly appropriate for compositions containing such adjuvants. They are cross-linked with an allyl sucrose or with allyl pentaerythritol. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like.

A “protectant” as used herein, refers to an anti-microbiological active agent, such as for example Gentamycin, Merthiolate, and the like. In particular adding a protectant is most preferred for the preparation of a multi-dose composition. Those anti-microbiological active agents are added in concentrations effective to prevent the composition of interest from any microbiological contamination or for inhibition of any microbiological growth within the composition of interest.

Moreover, this method can also comprise the addition of any stabilizing agent, such as for example saccharides, trehalose, mannitol, saccharose and the like, to increase and/or maintain product shelf-life.

PCV2 ORF2 DNA, as used herein and also as used within the processes provided herein is a highly conserved domain within PCV2 isolates and thereby, any PCV2 ORF2 would be effective as the source of the PCV ORF2 DNA. A preferred ORF 2 sequence is provided herein as SEQ ID NO. 7.

Amino acid sequences generated using methods of the present invention are preferably identical to the native or “naturally occurring” sequences. “Naturally occurring sequences are the sequences found in their natural state. For example, naturally occurring PCV2 ORF2 DNA would be the DNA sequence found when sequencing a full length PCV2 ORF2 sequence isolated from or identified from PCV2 in a porcine animal. However, it is understood by those of skill in the art that such sequences could be modified or vary by as much as 20% in sequence homology in comparison to the native sequence and still retain the antigenic characteristics that render them useful in immunogenic compositions. Of course, it is preferable that the variation be less than 15%, still more preferably as little as 6-10%, and even more preferably less than 5%, still more preferably less than 4%, even more preferably less than 3%, still more preferably less than 2%, and most preferably less than 1% in comparison to the native sequence. The antigenic characteristics of an immunological composition can be estimated by conventional methods known in the art. Moreover, the antigenic characteristic of a modified antigen is still retained, when the modified antigen confers at least 70%, preferably 80%, more preferably 90% of the protective immunity as compared to the antigen in its native or naturally occurring form. As such, protective immunity will generally result in a decrease or reduction in the incidence of or severity of clinical, pathological, and/or histopathological signs of infection by a pathogen. “Decrease” or “reduction in the incidence of or severity of clinical, pathological, and/or histopathological signs” shall mean that clinical signs are reduced in incidence or severity in animals receiving an administration of the expressed amino acid sequence in comparison with a “control group” of animals when both have been infected with the pathogen from which the expressed amino acid sequence is derived and wherein the control group has not received an administration of the expressed sequence. In this context, the term “decrease” or “reduction” means a reduction of at least 10%, preferably 25%, even more preferably 50%, most preferably of more than 100% as compared to the control group as defined above. An “immunogenic composition” as used herein, means an amino acid sequence or protein which elicits an “immunological response” in the host with a cellular and/or antibody-mediated immune response to such protein or amino acid. Preferably, this immunogenic composition is capable of conferring protective immunity against infection against a selected pathogen and the clinical signs associated therewith. In some forms, immunogenic portions of the native amino acid sequences or protein are used as the antigenic component in such compositions. The term “immunogenic portion” as used herein refers to truncated and/or substituted forms, or fragments of the native protein and/or polynucleotide, respectively. Preferably, such truncated and/or substituted forms, or fragments will comprise at least 8 contiguous amino acids from the full-length polypeptide. More preferably, the truncated or substituted forms, or fragments will have at least 10, more preferably at least 15, and still more preferably at least 19 contiguous amino acids from the full-length naturally occurring polypeptide. It is further understood that such sequences may be a part of larger fragments or truncated forms.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxyl terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Those of skill in the art will understand that compositions described herein may incorporate known, injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present invention can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants, are those described above.

The immunogenic compositions can further include one or more other immunomodulatory agents such as, e. g., interleukins, interferons, or other cytokines. The immunogenic compositions can also include Gentamicin and Merthiolate.

A further aspect relates to a container comprising at least one dose of an immunogenic composition of protein as provided herewith. Said container can comprise from 1 to 250 doses of the immunogenic composition, preferably it contains 1, 10, 25, 50, 100, 150, 200, or 250 doses of the immunogenic composition of desired protein or amino acid sequence.

A further aspect relates to a kit, comprising any of the containers, described above, and an instruction manual, including the information for the administration of at least one dose of the immunogenic composition of protein into an animal in need thereof. Moreover, according to a further aspect, said instruction manual comprises information regarding second or further administration(s) of at least one dose of the immunogenic composition of the amino acid or protein. Preferably, said instruction manual also includes the information, to administer an immune stimulant. “Immune stimulant” as used herein, means any agent or composition that can trigger a general immune response, preferably without initiating or increasing a specific immune response, for example the immune response against a specific pathogen. In preferred kits, it is further instructed to administer the immune stimulant in a suitable dose. Moreover, the kit may also comprise a container, including at least one dose of the immune stimulant.

However it is herewith understood, that antigens produced using the methods of the present invention refer to any composition of matter that comprises at least one antigen that can induce, stimulate or enhance the immune response against infection associated with said antigen, when administered to an animal in need thereof. The terms “immunogenic protein”, “immunogenic polypeptide” or “immunogenic amino acid sequence” as used herein refer to any amino acid sequence which elicits an immune or immunological response in a host against a pathogen comprising said immunogenic protein, immunogenic polypeptide or immunogenic amino acid sequence. An “immunogenic protein”, “immunogenic polypeptide” or “immunogenic amino acid sequence” as used herein, includes the full-length sequence of any proteins, analogs thereof, or immunogenic fragments thereof. By “immunogenic fragment” is meant a fragment of a protein which includes one or more epitopes and thus elicits the immunological response against the relevant pathogen. In one preferred embodiment of the present invention, immunogenic fragments of protein based antigen are attached to the sequence of the ORF 2 sequence. These protein-based antigens are preferably at least 8 amino acids in length, and more preferably between 8 and 200 amino acids in length. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998.

An “immunological or immune response” to a composition or vaccine is the development in the host of a cellular and/or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the incidence of or severity of up to and including a complete lack of the symptoms (clinical, pathological, and histopathological) associated with host infections as described above.

An “immunological active component” as used herein means a component that induces or stimulates the immune response in an animal to which said component is administered. According to a preferred embodiment, said immune response is directed to said component or to a microorganism comprising said component. According to a further preferred embodiment, the immunological active component is an attenuated microorganism, including modified live virus (MLV), a killed-microorganism or at least an immunological active part of a microorganism.

“Immunological active part of a microorganism” as used herein means a protein-, sugar-, and or glycoprotein containing fraction of a microorganism that comprises at least one antigen that induces or stimulates the immune response in an animal to which said component is administered. According to a preferred embodiment, said immune response is directed to said immunological active part of a microorganism or to a microorganism comprising said immunological active part.

In addition, the immunogenic and vaccine compositions of the present invention can include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like.

The composition according to the invention may be applied intradermally, intratracheally, or intravaginally. The composition preferably may be applied intramuscularly or intranasally. In an animal body, it can prove advantageous to apply the pharmaceutical compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months, and in different dosages.

“Foreign” amino acid segments or “foreign” DNA segments shall refer to such segments that are derived from different species. For example, the CSL 30 mer is “foreign” to the PCV2 ORF2 and foreign to the baculovirus.

“Amino terminus” or “carboxyl terminus” shall mean the amino end or carboxyl end, respectively. In the context of amino acids, any foreign amino acid segment attached to the amino or carboxyl end shall be at prior to the first or after the last amino acid of the non-foreign sequence. For example, if the M2ae1 segment is attached to the amino end of PCV2 ORF2, the M2ae1 segment shall appear prior to the first amino acid of PCV2 ORF2, as shown in the accompanying figures.

When a segment is “derived from” or “associated with” a known pathogen, this shall refer to the origin of the segment. For example, the CSL 30 mer is “derived from” or “associated with” Cryptosporidium parvum and the M2ae1 is “derived from” or “associated with” swine influenza.

“Induces” or “elicits” shall mean causes. For example, administration of the CSL 30 mer “induces” or “elicits” an immune response in the animal receiving such an administration.

“Clinical” signs shall refer to signs of infection from a pathogen that are directly observable from a live animal such as symptoms. Representative examples will depend on the pathogen selected but can include things such as nasal discharge, lethargy, coughing, elevated fever, weight gain or loss, dehydration, diarrhea, swelling, lameness, and the like.

“Pathological” signs shall refer to signs of infection that are observable at the microscopic or molecular level, through biochemical testing, or with the naked eye upon necropsy.

“Histopathological” signs shall refer to signs of tissue changes resulting from infection.

Thus, one aspect of the invention provides an immunogenic composition comprising an amino acid segment from PCV2 and a foreign amino acid segment attached to the PCV2 amino acid segment wherein the foreign amino acid segment is from an organism other than PCV2. One preferred PCV2 amino acid segment includes open reading frame 2, or an immunogenic portion thereof. In preferred forms, the PCV2 amino acid segment has at least 80% sequence homology with SEQ ID NO. 7. Preferably, the foreign amino acid segment is derived from a pathogen that produces clinical, pathological, and/or histopathological signs of infection after administration to an animal. Even more preferably, the foreign amino acid sequence is an antigen associated with or derived from a known pathogen. Advantageously, the foreign amino acid segment is detectable separately from said PCV2 amino acid segment, meaning that detection systems, assays, monoclonal antibodies, immunoblots, and the like are able to identify the presence of the foreign amino acid segment in the presence of the PCV2 amino acid segment as well as discern, or differentiate between the PCV2 amino acid segment and the foreign amino acid segment. Preferably, the foreign amino acid segment is detectable by an assay or test specific for the foreign amino acid segment. One preferred assay or test comprises monoclonal antibodies specific for the foreign amino acid segment. Preferably, the foreign amino acid segment retains at least 80% of its immunological properties in comparison to the same amino acid segment that is not attached to the PCV2 amino acid segment. The composition is characterized in that it is capable of inducing an immunological response in an animal receiving an administration thereof. This immunological response can be specific to the foreign amino acid segment, or to the PCV2 amino acid segment, or both. Preferably, the immunological response is sufficient to reduce the incidence of or lessen the severity of clinical, pathological, and/or histopathological signs of infection. As evidenced by the examples herein, the attachment of the foreign amino acid sequence to the PCV2 sequence can be at the amino or carboxyl terminus of the PCV2 amino acid segment, or at any point between the amino and the carboxyl terminus of the PCV2 amino acid segment. Preferred foreign amino acid segments are derived from an organism selected from the group consisting of Cryptosporidium parvum, swine influenza, and combinations thereof. In preferred forms, the foreign amino acid segment retains the antigenic characteristics of the native sequence and has at least 80% sequence homology with a sequence selected from the group consisting of SEQ ID NOS. 1 and 6. Such a composition will reduce the incidence of or severity of infection by the organism from which the foreign amino acid segments are derived or associated with. For example, for SEQ ID NOS. 1 and 6, these foreign amino acid segments are derived from or associated with Cryptosporidium parvum and swine influenza, respectively and will reduce the incidence of or lessen the severity of Cryptosporidium parvum or swine influenza, depending on which SEQ ID NO. is administered to an animal. Advantageously, the incidence of or severity of PCV2 will also be decreased when the composition comprising the foreign amino acid segment and the PCV2 amino acid segment are left intact, or co-administered after the foreign segment is excised from the PCV2 segment. In some preferred forms, the composition will further comprise an ingredient selected from the group consisting of adjuvants, pharmaceutical acceptable carriers, protectants, stabilizing agents, and combinations thereof.

In another aspect of the present invention, an expression vector comprising vector DNA and DNA derived from a first organism species and DNA derived from a second organism species, wherein the first and second organism species are different from one another and different from the organism species from which the vector DNA was derived. In some preferred forms the expression vector is from a baculovirus. One embodiment of the present invention includes PCV2 as the first organism species. When PCV2 is the first organism species, one preferred DNA segment therefrom is PCV2 ORF2. In preferred forms, the PCV2 ORF2 DNA has at least 80% sequence homology with SEQ ID NO. 7. In another embodiment of the present invention, the DNA from the second organism species encodes an amino acid segment that induces an immunological response in an animal receiving an administration thereof. Any organism that is pathogenic to animals can be used for purposes of the present invention. Preferably, the organism will have an amino acid segment that induces an immunological response when administered to an animal in need thereof. Still more preferably, the amino acid sequence being expressed is an antigen associated with a known pathogen. In preferred forms, the immunological response will be effective at reducing the incidence of or severity of clinical, pathological, or histopathological signs of infection from the first organism species, the second organism species, as well as both species simultaneously. In one embodiment of the present invention, the DNA from the second species is selected from the group consisting of Cryptosporidium parvum, E. coli, and combinations thereof. Specific representative examples of the DNA segments from a second species encode an amino acid segment having at least 80% sequence homology with and retaining the antigenic characteristics of a native sequence selected from the group consisting of SEQ ID NOS. 1 and 6.

Another aspect of the present invention provides a method of producing antigen. Preferably, the method comprises the steps of combining DNA encoding the antigen with PCV2 DNA to produce a combined DNA insert and expressing the combined DNA insert in an expression system. In preferred forms, the antigen has a length of about 8-200 amino acids. Preferably, the antigen-encoding DNA that is combined with the PCV2 DNA is derived from an organism species different from PCV2. Any organism species can be used but preferably, the amino acid sequence being expressed is an antigen associated with a known pathogen. Representative examples include organism species selected from the group consisting of Cryptosporidium parvum, swine influenza, and combinations thereof. When Cryptosporidium parvum and swine influenza are used as the organism species, preferred amino acid segments will retain the antigenic characteristics of the native sequence and have at least 80% sequence homology with a sequence selected from the group consisting of SEQ ID NOS. 1 and 6. Preferred PCV2 sequences will include ORF2 and in particular, SEQ ID NO. 7 and sequences retaining the antigenic characteristics of the native sequence and having at least 80% sequence homology with SEQ ID NO. 7. One preferred expression system comprises a Baculovirus expression system.

Another aspect of the present invention provides for the use of an antigen expressed by the above-described method in a vaccine or an immunogenic composition. Preferably, the antigen is associated with a known pathogen. Such an immunogenic composition or vaccine could further comprise an ingredient selected from the group consisting of adjuvants, pharmaceutical acceptable carriers, protectants, stabilizing agents, and combinations thereof.

A still further aspect of the present invention provides for the use of PCV2 ORF2 as a virus-like particle.

### DESCRIPTION OF THE DRAWING FIGURES

FIG. 1a is a photograph of SF cells infected with ORF2-CSL baculovirus stained with rabbit ant-CSL serum;

FIG. 1b is a photograph of SF cells infected with ORF2-CSL baculovirus stained with swine anti-PCV2 serum;

FIG. 1c is a photograph of SF cells infected with ORF2-CSL baculovirus stained with goat anti-rabbit-FITC;

FIG. 2 is a SDS page analysis verifying expression of ORF2-CSL from baculovirus infected cells;

FIG. 3 is the spot blot results for ORF2, CSL peptide, and ORF2-CSL in anti-PCV2, rabbit pre-immune day 0 serum, and rabbit post-immune day 84 serum;

FIG. 4a is a photograph of a Western blot showing the ORF2-CSL protein;

FIG. 4b is a Coomasie stained blot showing ORF2, and ORF2-CSL proteins;

FIG. 5 is a comparison of the PCV2 ORF2 sequence with and without the internal AscI restriction site;

FIG. 6 is a comparison of the PCV2 ORF2 sequence with and without the internal M2ae1 amino acid sequence; and

FIG. 7 is a comparison of the PCV2 ORF2 sequence with and without the amino M2ae1 amino acid sequence.

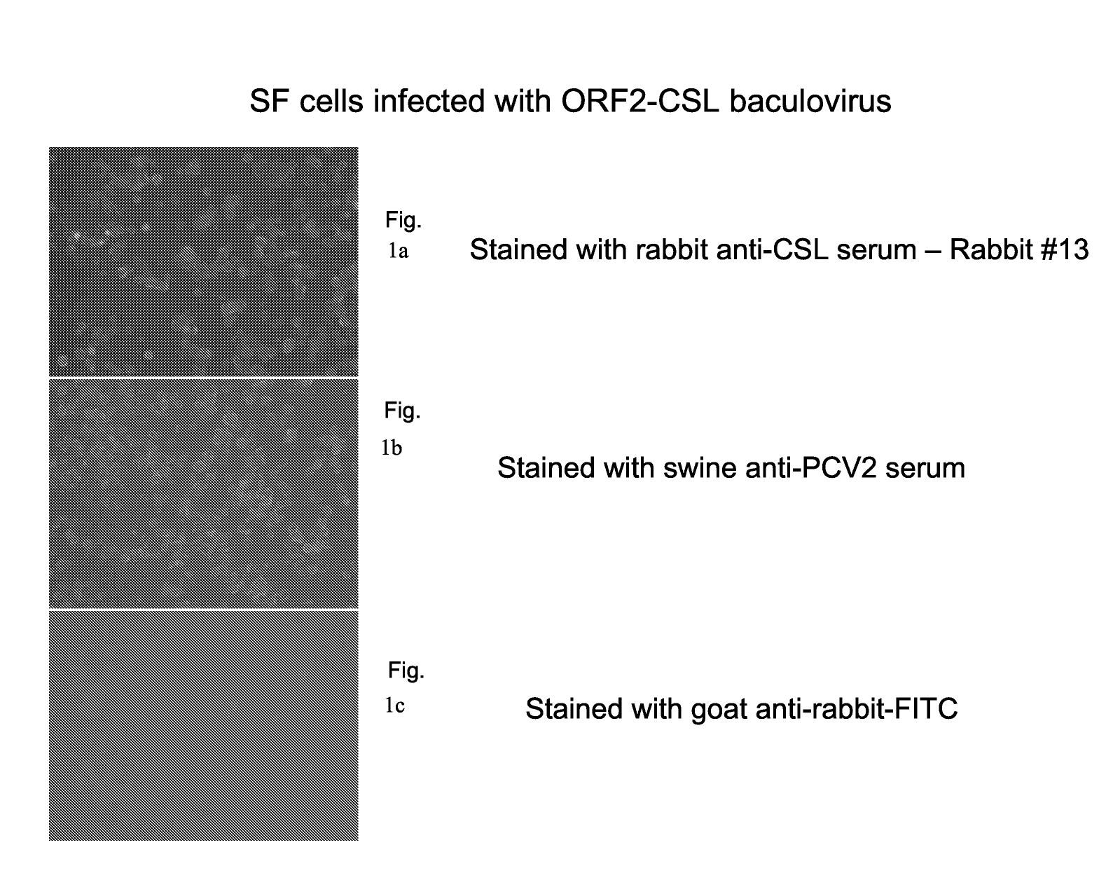

### DETAILED DESCRIPTION OF THE PREFERRED EMBODIMENTS

The following examples set forth preferred materials and procedures in accordance with the present invention. It is to be understood, however, that these examples are provided by way of illustration only, and nothing therein should be deemed a limitation upon the overall scope of the invention.

### EXAMPLE 1

### Materials and Methods:

### CSL 30-mer Reverse Translation

The amino acid sequence of the CSL 30-mer peptide is AINGGGATLPQKLYLTPNVLTAGFAPYIGV (SEQ ID NO. 1). This 30-mer amino acid sequence was reverse translated into nucleotide sequence using the optimal codon usage for Drosophila. A complementary primer sequence matching the 3′ end of the ORF2 gene plus the nucleotide sequence for the CSL 30-mer was synthesized.

### Primer Design

PCV2-5-HA primer
(SEQ ID NO. 2)
5′-TGGATCCGCCATGACGTATCC-3′
(PCV2 ORF2 ATG start site is underlined)
L-PCV2CSL primer
(SEQ ID NO. 3)
5′-AGATCTACACGCCGATGTAGGGGGCGAAGCCGGC
GGTCAGCACGTTGGGGGTCAGGTACAGCTTCTGGGGCAGG
GTGGCGCCGCCGCCGTTGATGGCGGGTTCAAGTGGGGGGT
CTTTAA-3′

PCR of PCV2 ORF2 with C-Terminal CSL Tail

The PCV2 ORF2 gene that had been previously cloned in the pGEM-T Easy plasmid (Promega) served as the template for the PCR reaction, and was mixed with Amplitaq Gold (Applied Biosystems) and the PCV2-5-HA and L-PCV2CSL primers. The PCR reaction was heated to 94° C. for 10 minutes. The PCR reaction then proceeded through 40 cycles of 94° C. for 30 seconds, 40° C. for 30 seconds, and 72° C. for 1 minute. The PCR cycle was completed following a final cycle of 72° C. for 10 minutes. The PCV2 ORF2 CSL PCR product was visualized by agarose gel electrophoresis. The PCR product was purified from the gel and ligated into the pGEM-T Easy cloning vector, transformed into DH5 α E. coli competent cells, and screened for ampicillin resistance. Transformed colonies were used to inoculate 3 ml of LB broth with ampicillin and grown overnight at 37° C. A 1.5 ml aliquot of the overnight culture was harvested by centrifugation and plasmid DNA extracted by the Qiagen Mini-Prep plasmid kit. The purified plasmid DNA was then verified by dideoxynucleotide sequencing.

The PCV2 ORF2 CSL gene was excised from the pGEM-T Easy plasmid by digestion with the restriction enzymes BamHI and NotI and ligated into the baculovirus transfer vector, pVL1393. The resulting PCV2 ORF2 CDL/pVL1393 plasmid was then purified using the Qiagen Mini-Prep plasmid kit for subsequent use in transfections.

### Generation of Recombinant Baculovirus Containing the PCV2 ORF2 CSL Gene

The PCV2 ORF2 CDL/pVL1393 plasmid and the DiamondBac® linearized baculovirus DNA (Sigma) were cotransfected into Sf9 insect cells using the ESCORT transfection reagent (Sigma) for 5 hours at 27° C. The transfection medium was removed and the transfected cells were then gently washed, replenished with media, and incubated at 27° C. Five days later, the cell supernatant containing the generated recombinant baculovirus was harvested and stored at 4° C. The remaining transfected Sf9 cells were fixed with acetone: methanol and used in immunofluorescence assay (IFA) with swine anti-PCV2 antiserum to verify expression of PCV2 ORF2 in the transfected cells.

The harvested PCV2 ORF2 CSL recombinant baculovirus supernatant was plaque purified on Sf9 cells prior to generation of virus stocks.

Visualization and Immunological Detection of the Chimeric PCV2 ORF2 CSL (PCV2 ORF2 Gene Fused with the CSL 30-mer as a C-Terminal Tail).

IFA was performed on transfected Sf9 cells for detection of PCV2 ORF2, H5HA or H7HA antigen. Materials included a fixed 6-well plate, swine anti-PCV2, chicken anti-H5 and anti-H7, goat-chicken FITC, goat α-chicken FITC, rabbit α-swine FITC, 1×PBS, and glycerol (50:50). Sf9 cells were fixed in a 6-well plate and rinsed with 1×PBS. Two ml of PBS was left on each well. Twenty microliters of swine anti-PCV2 was added to untransfected Sf9 cell wells, the PCV2 ORF2-HA transfected well, and the PCV2 ORF2 CSL transfected well. The alpha PCV2 serum was at a 1:100 dilution. The plate was swirled to mix. A 1:100 dilution of chicken α-H5 and α-H7 was added to the untransfected Sf9 cell well, the H7HA transfected well, the H7HA transfected well, and the H5HA transfected well, as above. The plate was again swirled to mix. The plate was incubated at 37° C. for one hour. The primary antibody solution was removed. Next, the well was washed three times with PBS and removed for the final wash. Two ml's of PBS was added to each well. Next, twenty ml's of rabbit α-swine was added to the wells with primary PCV2 antisera, and mixed together. Next, twenty ml's of goat α-chicken FITC was added to the wells and treated with primary chicken sera and mixed. These were incubated at 37° C. for one hour. The FITC solution was removed and they were washed three times with PBS. The final wash was removed. Next, one ml of the glycerol was added to each well and the excess was removed by a flick of the wrist. Each well was then observed for specific fluorescence. The results indicate that H5HA recombinant baculovirus was generated, as was the PCV2 ORF2-HA and CSL recombinant.

### EXAMPLE 2

Expression Analysis of ORF2-CSL from Baculovirus-Infected SF+ Cells (Sample 070).

### Materials and Methods:

Samples (pellet and supernatant) of baculovirus-infected SF+ cells were collected at 96, 120, and 144 hours for analysis of ORF2-CSL from these baculovirus-infected SF+ cells. The following procedure was applied to each sample: The SF+ cell samples were thawed and the supernatant was removed. The pellet was resuspended in 200 μl of 100 mM NaHCO₃, pH 8.3, and pipetted up and down to mix. The sample was then allowed to sit for 30 min at room temperature (about 25-30° C.). The sample was then centrifuged for 2 minutes at 20,000×g at 4° C. The supernatant and bicarbonate lysate of pellet were separate and the entire sample was stored on ice at about 4° C.

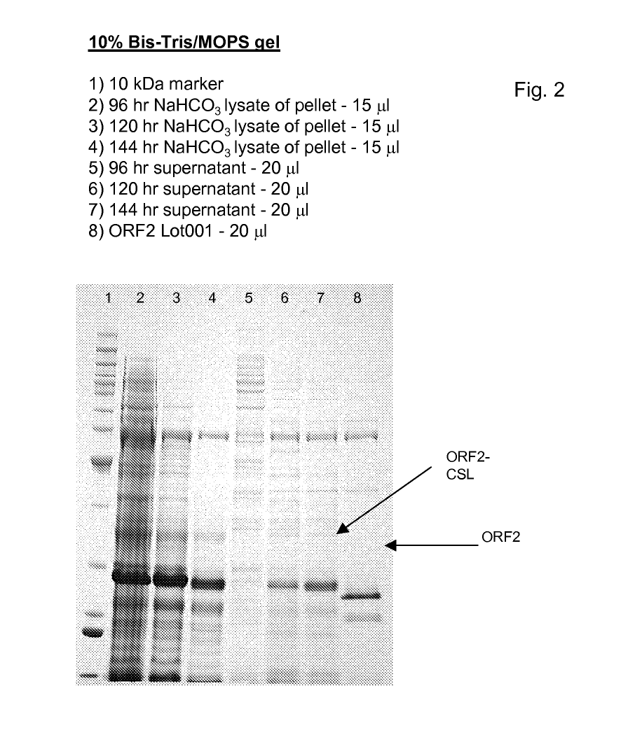

The bicarbonate lysate of the pellet and the supernatant samples were then subjected to SDS Polyacrylamide Cell Electrophoresis (SDS-PAGE) analysis on 10% Bis-Tris gel in MOPS buffer. 15 μl of each pellet bicarbonate lysate and 20 μl of each supernatant sample were loaded onto the gel in their respective assigned lanes. Lane 1 contained the 10 kDa marker. Lane 2 contained the NaHCO₃ lysate of pellet from the 96 hr sample. Lane 3 contained the NaHCO₃ lysate of pellet from the 120 hr sample. Lane 4 contained the NaHCO₃ lysate of pellet from the 144 hr sample. Lane 5 contained the supernatant from the 96 hr sample. Lane 6 contained the supernatant from the 120 hr sample. Lane 7 contained the supernatant from the 144 hr sample. And Lane 8 contained 20 μl of unaltered ORF2 to act as the control. The results for the gel are depicted in FIG. 2.

### Results:

Lanes, 2, 3, and 4, which contained the bicarbonate lysate of pellets from the 96, 120 and 144 hour samples respectively, clearly demonstrated the expression of ORF2-CSL. The results suggest that ORF2-CSL is indeed expressed from the new baculovirus construct. ORF2-CSL is estimated to be about 3 kDa larger than ORF2, and the molecular weight observed, is consistent with that estimation. Additionally, ORF2-CSL was observed to be present in Lanes 6, and 7, which contained the supernatant of the 120 hr and 144 hr samples, with the presence being much stronger in the 144 hr sample. The fact that ORF2-CSL was observed emerging over time in the supernatant suggests that the virus-like-particle (VLP) structure of ORF2 is still largely intact.

### EXAMPLE 3

### Materials and Methods:

This example demonstrates Spot Blot analysis of ORF2, ORF2-CSL, and CSL peptide with pre- and post-immune rabbit serum, and swine anti-PCV2 serum. Three protein samples were used in this example: Standard ORF2 protein, CSL peptide and ORF2-CSL bicarbonate lysate pellet from baculovirus-infected SF+ cells 144 hr post infection. 5 μl of each protein sample was spotted onto a piece of nitrocellulose in a row, and each spot was labeled. This process was repeated three times, resulting in three identically spotted pieces of nitrocellulose containing one spot from each protein sample (three spots total). Each spot blot was allowed to dry, and then was incubated for at least 1 hr in about 50 ml TTBS+2% dry milk (w/v). The membranes were then incubated with the primary antibodies. The first nitrocellulose piece was incubated (blotted) with swine anti-PCV2 serum diluted 1:100 in TTBS+2% dry milk for 1 hour. The second nitrocellulose piece was incubated (blotted) with rabbit pre-immune serum diluted 1:200 in TTBS+2% dry milk for 1 hour. The third nitrocellulose piece was incubated (blotted) with rabbit post-immune serum diluted 1:200 in TTBS+2% dry milk for 1 hour.

Each blot was washed three times for two minutes with TTBS (1× TBS plus 0.05% Tween20, prepared fresh). The TBS wash was formulated by adding 200 ml 1 M Tris, pH 8 to 292.2 g NaCl, the pH was adjusted to 7.4 with HCl, the solution was brought to a total volume of 1 L by adding water (qs), and the filter was sterilized. After washing, the membranes were then incubated with secondary antibodies. The first nitrocellulose piece was incubated with goat anti-swine-HRP diluted 1:1000 in TTBS+2% dry milk for 1 hour. The second nitrocellulose piece was incubated with goat anti-rabbit-HRP diluted 1:1000 in TTBS+2% dry milk for 1 hour. The third nitrocellulose piece was incubated with goat anti-rabbit-HRP diluted 1:1000 in TTBS+2% dry milk for 1 hour. Each blot was then washed two times for two minutes with TTBS, and then washed one time for two minutes with PBS (10×PBS 1 L). The PBS wash was formulated by adding 0.96 g NaH₂PO₄ (monobasic) anhydrous to 13.1 g NaH₂PO₄ (dibasic) anhydrous and 87.7 g NaCl, the mixture was dissolved in water, and the pH was adjusted to 7.4 with HCL, the solution was qs to 1 L, and the filter was sterilized.

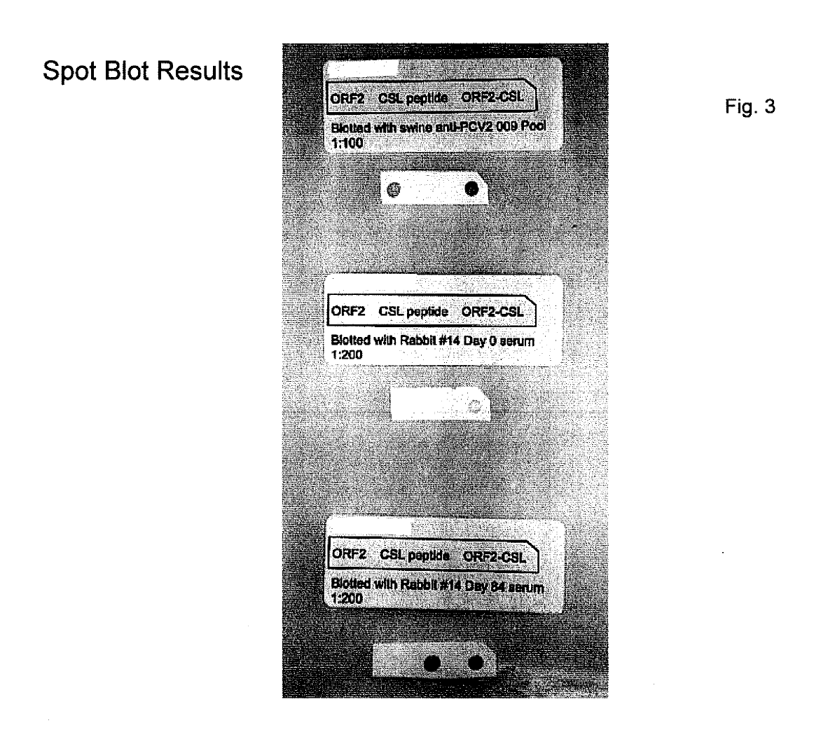

10 ml of Opt-2CN substrate was then added to each blot, and allowed to develop for less than about 5 minutes. The blots were each rinsed with water to stop the process, and analyzed. Results for the Spot Blot can be seen in FIG. 3.

### Results:

Both ORF2 and ORF2-CSL reacted with swine anti-PCV2 serum, on the first nitrocellulose piece, indicating that the ORF2 portion of ORF2-CSL is structurally intact. The results from the second nitrocellulose piece indicate that ORF2-CLS also reacts with rabbit pre-immune serum. The results from the third nitrocellulose also suggest that the CSL portion of ORF2-CSL is as expected (structurally intact) because it reacts with rabbit post-immune serum. The reactivity with CSL peptide by rabbit post-immune serum and not pre-immune serum or anti-PCV2 serum also confirms the specificity of the CSL reactivity in the post-immune serum. Taken together, these results strongly indicate that the CSL peptide is being expressed as a fusion protein with ORF2.

### EXAMPLE 4

### Western Blot

### Materials and Methods:

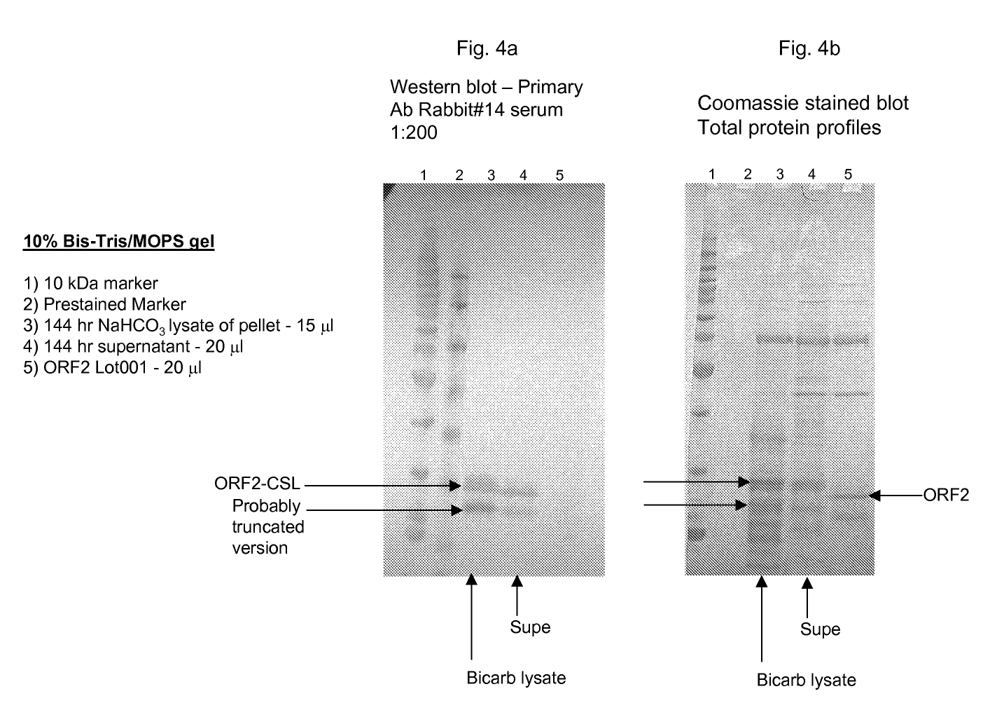

This Example demonstrates Western Blot analysis of ORF2 and ORF2-CSL pellet and supernatant from baculovirus-infected SF+ cells 144 hr post infection with rabbit post-immune serum. The protein samples were subjected to SDS-PAGE analysis on 10% Bis-Tris gel in MOPS buffer. Two replicate sample sets were run on the same gel. Lane 1 contained 10 kDa marker. Lane 2 contained a pre-stained marker. Lane 3 contained ORF2-CSL bicarbonate lysate pellet from 144 hr post infection baculovirus infected SF+ cells. Lane 4 contained ORF2-CSL supernatant from the same sample. Lane 5 contained standard ORF2 protein sample. After SDS-PAGE, the proteins were transferred electrophoretically (30 V constant for more than about 1 hour) from the gel to a Polyvinylidene Difluoride (PVDF) membrane in a Novex Blot Module (Novex; San Diego, Calif.). After transblotting, the sample lanes were incubated at least 1 hour in about 50 ml TTBS+2% dry milk. The blot was then cut into two replicate blots. One was incubated/blotted with the primary antibody, rabbit post-immune serum diluted 1:200 in TTBS+2% dry milk for one hour, and the other was dried and stained to show total protein profiles. Results for the second blot can be seen in FIG. 4B.

The first blot was then washed three times for 2 minutes with TTBS (1× TBS plus 0.05% Tween20, prepared fresh). The blot was then incubated with the secondary antibody, goat anti-rabbit-HRP diluted 1:1000 in TTBS+2% dry milk for 1 hour. After incubation, the blot was washed two times for two minutes with TTBS (1× TBS plus 0.05% Tween20, prepared fresh) and one time for two minutes with PBS. The blot was then visualized using 10 ml Opti-4CN substrate, allowing the blot to develop for less than about 5 minutes. The blot was rinsed with water to stop the process and analyzed. Results for this blot can be seen in FIG. 4A.

### EXAMPLE 5

This example generates a PCV2 ORF2 VLP with an in-frame insertion of 24 amino acids of the Influenza M2ae region.

### Materials and Methods:

M2ae1 24-mer with AscI

The amino acid sequence of the M2ae1 24-mer is MSLLTEVETPIRNEWGCRCNDSSD (SEQ ID NO. 6). The M2ae1 24 amino acid sequence was reverse translated into its nucleotide sequence using the optimal codon usage for Drosophila. PCR was performed to add flanking AscI restriction enzyme sites to the M2ae1 coding region.

Introduction of AscI Restriction Site into PCV2 ORF2 Coding Region

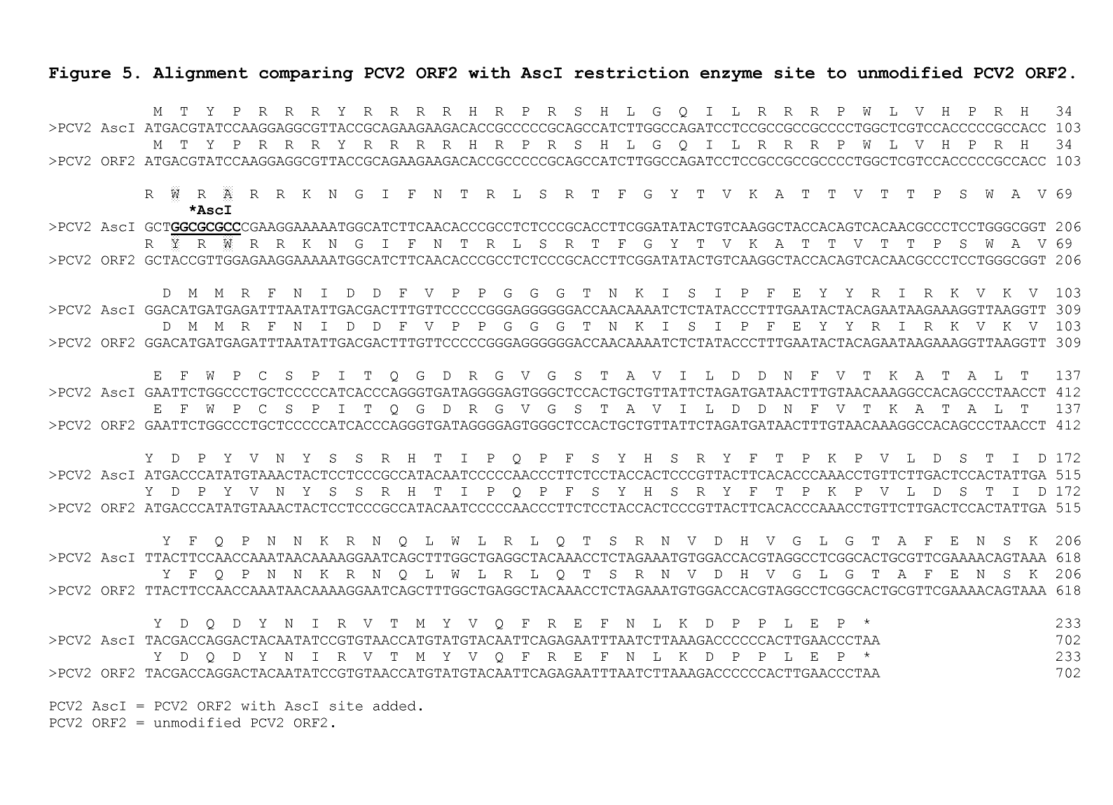

Using site-directed mutagenesis, the AscI restriction enzyme site was introduced into the coding region of PCV2 ORF2 (SEQ ID NO. 7) (refer to FIG. 5). As shown in FIG. 5, the introduction of the AscI site introduced two amino acid changes into the PCV2 ORF2 coding region, Y36W (Tyrosine to Tryptophan at amino acid position 36, replacing a neutral polar amino acid with another neutral polar amino acid) and W38A (Tryptophan to Alanine at amino acid position 38, replacing a neutral polar amino acid with a neutral non-polar amino acid) (SEQ ID NO. 8).

Insertion of M2ae1 into the PCV2 ORF2 Coding Region

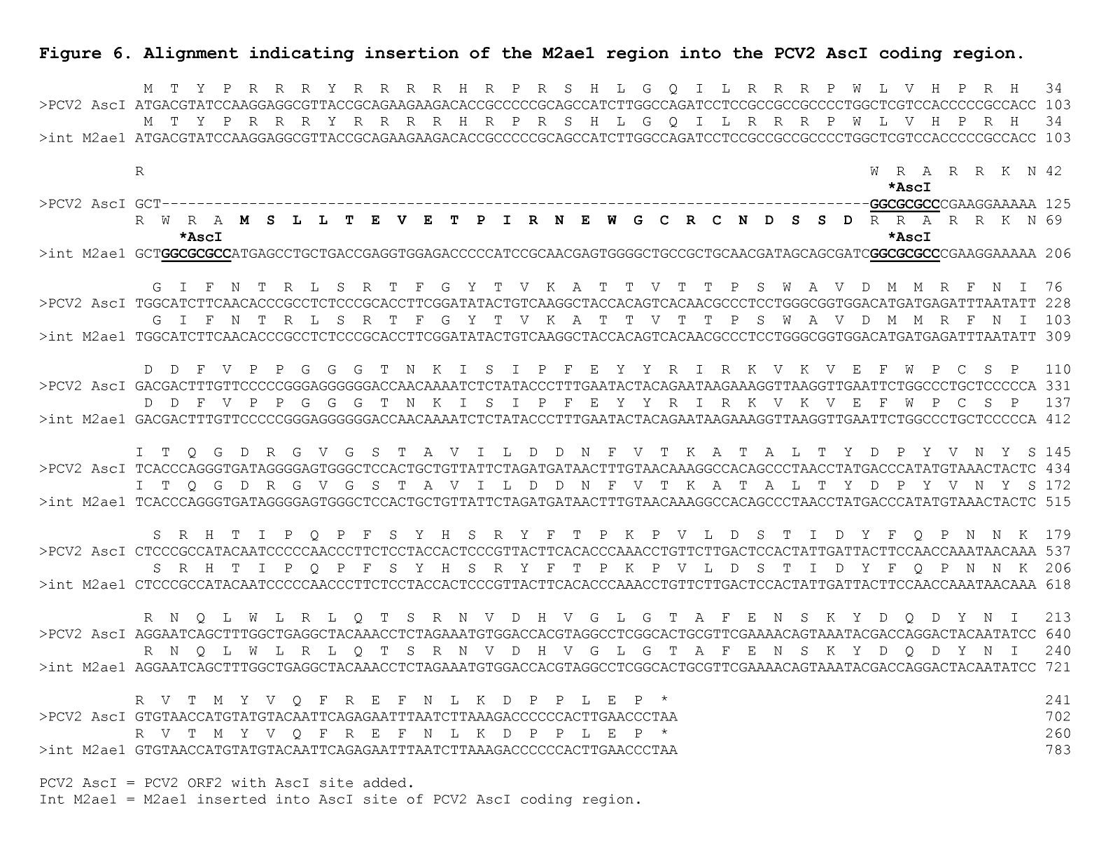

Refer to FIG. 6 for a representation of the insertion of the M2ae1 region into PCV2 ORF2 (SEQ ID NO. 9). Briefly, using standard molecular biology methods, the M2ae1-AscI region was cloned into the AscI site of the PCV2 ORF2 gene in the baculovirus transfer vector, pVL1393. The resulting PCV2 ORF2 internal M2ae1/pVL1393 (designated as A-34) plasmid was then purified using the Qiagen Mini-Prep plasmid kit for subsequent use in transfection.

Generation of Recombinant Baculovirus Containing the PCV2 ORF2 with Internal M2ae1

The A-34 PCV2 ORF2 internal M2ae1/pVL1393 plasmid and the DiamondBac® linearized baculovirus DNA (Sigma) were cotransfected into Sf9 insect cells using the ESCORT transfection reagent (Sigma) for 5 hours at 28° C. The transfection medium was removed and the transfected cells were then gently washed, replenished with media, and incubated at 27° C. Five days later, the cell supernatant containing the generated recombinant baculovirus was harvested and stored at 4° C. The remaining transfected Sf9 cells were fixed with acetone: methanol and used in immunofluorescence assay (IFA) with the anti-Influenza A M2 monoclonal antibody 14C2 to verify the expression of the M2ae1 region transfected Sf9 cells. The sequence of the expressed chimeric protein comprising PCV2 ORF2 and the internal M2ae1 segment is provided herein as SEQ ID NO. 11.

The harvested A-34 M2ae1 ORF2 PCV2 Baculovirus DB supernatant was subsequently purified by limiting dilution on Sf9 cells prior to generation of virus stock material.

Immunological Detection of PCV2 ORF2 with Internal M2ae1

Verification of M2ae1 expression in A-34 M2ae1 ORF2 PCV2 Baculovirus DB-infected Sf9 cells was previously confirmed by IFA. However, as a means to further confirm the expression of M2ae1 along with PCV2 ORF2, an immunoblot on PCV2 ORF2 internal M2ae1 harvested supernatant from baculovirus-infected insect cell cultures was performed.

Briefly, harvested supernatant from baculovirus-infected insect cell cultures was blotted onto PVDF membranes and the presence of PCV2 ORF2 and/or M2ae1 antigens were tested in an immunoblot. The primary antibodies used for immunoblot detection of PCV2 ORF2 were the anti-PCV2 ORF2 monoclonal antibody 6C4-2-4A3-5D10 and purified swine anti-PCV2 ORF2 IgG. The primary antibodies used for immunoblot detection of M2ae1 were the anti-M2 monoclonal antibody 14C2 (Santa Cruz Biotechnology, Inc.) and swine anti-M2aeC5 serum. The respective secondary antibodies used in the immunoblot were HRP-labeled goat anti-mouse conjugate and goat-anti-swine conjugate. Opti-4CN substrate (BioRad) was used for colorimetric detection on the immunoblots. The immunoblots revealed the presence of the PCV2 ORF2 and M2ae1 antigens.

This materials and methods used for this example are described in greater detail below:

### Materials and Methods:

For plasmid purification, the QlAprep Spin MiniPrep (QIAGEN, Gaithersburg, Md.) was used and manufacturer's protocol was followed. Briefly, 1.5 ml of culture was pelleted for 1 minute at 14,000 rpm. The supernatant was discarded before repeating the pelleting procedure and discarding the supernatant again. The pellet was reconstituted in 250 μl of buffer P1 and added to 250 μl of buffer P2, which was then mixed by inversion. Next, 350 μl of buffer N3 was added and mixed by inversion before being spun at 14,000 rpm for 10 minutes. The supernatant was transferred to the QlAprep spin column in a collection tube, spun at 14,000 rpm for 60 seconds, the flow through was discarded and the column reassembled. Next, 750 μl of buffer PE was added and spun at 14,000 for 60 seconds, the flow through was discarded and the column reassembled. The column was spun at 14,000 rpm for 1 minute in order to dry it, and then the column was transferred to a new 1.5 ml tube. Finally, 50 μl of H₂O was added, incubated at room temperature for 1 minute and then spun at 14,000 rpm for 1 minute before discarding the column.

To cut, purify, and ligate the M2ae1 fragment, a restriction digestion was performed using New England Biolabs (Ipswich, Mass.) product and procedure. Briefly, 6 μl of New England Biolabs Buffer 4, 49 μl of DNA, and 5 μl of AscI was mixed together in a 600 μl centrifuge tube. The tube was incubated at 37° C. for 1 hour before adding 3 μl of 6× loading dye to each tube and shaking well. The reactions were then loaded on a 1.5% agarose gel that was run at about 100 volts for 60 minutes before photographing or scanning the gel. The desired band was excised from the gel and placed in a 1.5 ml centrifuge tube before adding 10 μl of Membrane Binding solution per 10 mg of gel slice. This was vortexed and incubated at 50-65° C. until the gel was completely dissolved. The mini column was inserted into a collection tube and the prepared DNA was transferred to the column assembly. This was incubated at room temperature for 1 minute and centrifuged at 14,000 rpm for 1 minute before discarding the flow through. The washing step consisted of adding 700 μl of membrane wash solution, centrifuging at 14,000 rpm for 1 minute, discarding the flow-through, adding 500 μl of the membrane wash solution, centrifuging at 14,000 for 5 minutes, discarding the flow through, and then recentrifuging for 1 minute at 14,000 rpm with the lid open to dry the membrane. The elution step consisted of transferring the mini column to a 1.5 ml centrifuge tube, adding 50 μl of nuclease-free H₂O, incubating at room temperature for 1 minute, centrifuging for 1 minute at 14,000 rpm, discarding the column, and storing at −20° C. for future use.

The Ligation reaction (1) was performed by mixing 1 μl of the ORF2-AscI PVL1393 vector, 7 μl of the M2ae1 insert, 1 μl of the 10× Ligation buffer, and 1 μl of T-4 DNA Ligase in a 0.5 ml microfuge tube. This was incubated over the weekend at 4° C.

The transformation of M2ae1/AscI-Orf2-PVL1393 (transformation 1) and religation of the M2ae1 segment was performed using conventional protocols. Briefly, Max Effc Competent DHSx Cells were thawed on ice and 50 μl per reaction was transferred to 17×100 mm pp Falcon tubes. The extra cells were refrozen in an EtOH/dry ice bath. Next, 2 μl of the ligation reaction 1 was added to the cells and incubated on ice for 30 minutes before heat shocking the cells at exactly 42° C. for exactly 45 seconds. The tubes were returned to ice for 2 minutes before adding 950 μl SOC and incubating at 37° C. for 1 hour with about 225 rpm shaking. Then, 50 and 200 μl aliquots were spread on LB and CIX which were inverted and incubated overnight at 37° C. overnight.

The Ligation reaction (2) to religate the M2ae1 was performed by mixing 1 μl of the AscI PVL1393 vector, 7 μl of the concentrated M2ae1 insert, 1 μl of the 10× Ligation buffer, and 1 μl of T-4 DNA Ligase in a 0.5 ml microfuge tube. This was incubated overnight at 4° C.

To transform the concentrated M2ae1/AscI-ORF2-PVL1393 into the cells, transformation reaction (2) was performed using conventional methods. Briefly, Max Effc Competent DHSx Cells were thawed on ice and 50 μl per reaction was transferred to 17×100 mm pp Falcon tubes. The extra cells were refrozen in an EtOH/dry ice bath. Next, 2 μl of the ligation reaction 2 was added to the cells and incubated on ice for 30 minutes before heat shocking the cells at exactly 42° C. for exactly 45 seconds. The tubes were returned to ice for 2 minutes before adding 950 μl SOC and incubating at 37° C. for 1 hour with about 225 rpm shaking. Then, 50 and 200 μl aliquots were spread on LB and CIX which were inverted and incubated overnight at 37° C. overnight.

To check the colonies for the desired clone, a PCR reaction was set up using the following parameters and reagents: 1 cycle at 95° C. for 5 minutes, 35 cycles at 95° C. for 15 seconds, 35 cycles at 50° C. for 15 seconds, 35 cycles at 72° C. for 60 seconds, 1 cycle at 72° C. for 5 minutes and 1 cycle at 4° C. for infinity; 12.5 μl of 2× Amplitaq Gold Mastermix, 11.5 μl of Rnase/Dnase free water, 0.5 μl of primer pv1-U, 0.5 μl of primer gel-scrnL, and the selected colony. The comb(s) were removed from a 48 well 2% agarose E-gel Cassette (Invitrogen). Exactly 10 μl of DEPC H₂O EMD was loaded into each well and 10 μl of DNA marker and 10 μl of sample containing 6× loading dye was added to the desired wells. The power button was pressed until the display read “EG.” Slide onto the E-Gel Mother base (a steady red light illuminates when inserted correctly) and press the power button again (the light will turn to green to indicate the gel is running). The gel was allowed to run for about 20 minutes. Selected colonies were then grown for MiniPrep by inoculating 3 ml of LB broth and 6 μl CAR stock with a loopful of the selected colonies. This was then incubated overnight at 37° C. with shaking at about 225 rpm.

The plasmid was then purified using the QlAprep Spin MiniPrep kit according to manufacturer's instructions and as described above.

To start overnight cultures of M2ae1/pGemT-easy selected colonies for plasmid purification and sequence analysis, a loopful of the selected colonies were then grown for MiniPrep by inoculating 3 ml of LB broth and 6 μl CAR stock with the selected colonies. This was then incubated overnight at 37° C. with shaking at about 225 rpm. These were purified as described above for the QlAprep Spin MiniPrep.

To excise the M2ae1 fragment, a restriction digestion was done according to New England Biolabs conventional procedure. Briefly, 5 μl of New England Biolabs Buffer 4, 25 μl of DNA, 5 μl of AscI, and 15 μl of H₂O was mixed together in a 600 μl centrifuge tube. The tube was incubated at 37° C. for 1 hour before adding 3 μl of 6× loading dye to each tube and mixing well. The reactions were then loaded on a 1.5% agarose gel that was run at about 100 volts for 60 minutes before photographing or scanning the gel.

Next, the M2ae1 fragment was gel purified according to the QIAEX II Agarose Gel Extraction protocol (QIAGEN, QIAEX II Handbook 02/99) and ligated into PVL1393 by ligation reaction (3). Briefly, to ligate the M2ae1 into PVL1393, 2 μl of the PVL1393 vector, 6 μl of the AscI concentrated M2ae1 insert, 1 μl of the 10× Ligation buffer, and 1 μl of T-4 DNA Ligase were mixed together in a 0.5 ml microfuge tube and incubated overnight at 4° C.

To transform the AscI-M2ae1/PVL1393 insert into DHSx cells, Max Effc Competent DHSx Cells were thawed on ice and 50 μl per reaction was transferred to 17×100 mm pp Falcon tubes. The extra cells were refrozen in an EtOH/dry ice bath. Next, 2 μl of the ligation reaction 2 was added to the cells and incubated on ice for 30 minutes before heat shocking the cells at exactly 42° C. for exactly 45 seconds. The tubes were returned to ice for 2 minutes before adding 950 μl SOC and incubating at 37° C. for 1 hour with about 225 rpm shaking. Then, 50 and 200 μl aliquots were spread on LB and CIX which were inverted and incubated overnight at 37° C. overnight.

To restriction cut the ORF2-AscI-PVL1393 with AscI, 2 μl of New England Biolabs Buffer 4, 2.5 μl of DNA, 2 μl of AscI, and 12.5 μl of H₂O was mixed together in a 600 μl centrifuge tube. The tube was incubated at 37° C. for 1 hour before adding 3 μl of 6× loading dye to each tube and mixing well. The reactions were then loaded on a 1.5% agarose gel that was run at about 100 volts for 60 minutes before photographing or scanning the gel.

Next, the gel was purified, and a dephosphoralation reaction and another ligation reaction were performed. The Gel purification followed the steps of the WIZARD SV Gel Clean-up. Briefly, the mini column was inserted into a collection tube and the prepared DNA was transferred to the column assembly. This was incubated at room temperature for 1 minute and centrifuged at 14,000 rpm for 1 minute before discarding the flow through. The washing step consisted of adding 700 μl of membrane wash solution, centrifuging at 14,000 rpm for 1 minute, discarding the flow-through, adding 500 μl of the membrane wash solution, centrifuging at 14,000 for 5 minutes, discarding the flow through, and then recentrifuging for 1 minute at 14,000 rpm with the lid open to dry the membrane. The elution step consisted of transferring the mini column to a 1.5 ml centrifuge tube, adding 50 μl of nuclease-free H₂O, incubating at room temperature for 1 minute, centrifuging for 1 minute at 14,000 rpm, discarding the column, and storing at −20° C. for future use. The dephosphoralation step comprised adding 2 μl of 10× SAP buffer to 16 μl of gel purified plasmid, then adding 2 μl of SAP before incubating at 37° C. for 15 minutes and then inactivating the SAP at 65° C. for 15 minutes. The ligation reaction was then performed by mixing 4 μl of the AscI-ORF2-PVL1393 vector, 12 μl of the AscI cut M2ae1 insert, 2 μl of the 10× Ligation buffer, and 2 μl of T-4 DNA Ligase were mixed together in a 0.5 ml microfuge tube and incubated overnight at 4° C.

To transform the ligated vector and insert it into competent DHSx's fopr propagation and future screening, Max Effc Competent DHSx Cells were thawed on ice and 50 μl per reaction was transferred to 17×100 mm pp Falcon tubes. The extra cells were refrozen in an EtOH/dry ice bath. Next, 2 μl of the ligation reaction 2 was added to the cells and incubated on ice for 30 minutes before heat shocking the cells at exactly 42° C. for exactly 45 seconds. The tubes were returned to ice for 2 minutes before adding 950 μl SOC and incubating at 37° C. for 1 hour with about 225 rpm shaking. Then, 50 and 200 μl aliquots were spread on LB and CIX which were inverted and incubated overnight at 37° C. overnight.

To screen the transformants for the presence of the desired insert, an Amplitaq Gold PCR reaction was performed using the following parameters and reagents: 1 cycle at 95° C. for 5 minutes, 35 cycles at 95° C. for 20 seconds, 35 cycles at 50° C. for 20 seconds, 35 cycles at 72° C. for 20 seconds, 1 cycle at 72° C. for 5 minutes and 1 cycle at 4° C. for infinity; 12.5 μl of 2× Amplitaq Gold Mastermix, 11.5 μl of Rnase/Dnase free water, 0.5 μl of primer pv1-U, 0.5 μl of primer ae1 scrnL, and the selected colony. The comb(s) were removed from a 48 well 2% agarose E-gel Cassette (Invitrogen). Exactly 7.5 μl of DEPC H₂O EMD was loaded into each well and 10 μl of DNA marker and 10 μl of sample containing 6× loading dye was added to the desired wells. The power button was pressed until the display read “EG.” Slide onto the E-Gel Mother base (a steady red light illuminates when inserted correctly) and press the power button again (the light will turn to green to indicate the gel is running). The gel was allowed to run for about 20 minutes. Selected colonies were then grown for MiniPrep by inoculating 3 ml of LB broth and 6μl CAR stock with a loopful of the selected colonies from the M2ae1-AscI/PVL1393 transformation. This was then incubated overnight at 37° C. with shaking at about 225 rpm. To purify the plasmids for sequence analysis, the QIAprep Spin MiniPrep was used according to manufacturer's instructions. Briefly, 1.5 ml of culture was pelleted for 1 minute at 14,000 rpm. The supernatant was discarded before repeating the pelleting procedure and discarding the supernatant again. The pellet was reconstituted in 250 μl of buffer P1 and added to 250 μl of buffer P2, which was then mixed by inversion. Next, 350 μl of buffer N3 was added and mixed by inversion before being spun at 14,000 rpm for 10 minutes. The supernatant was transferred to the QlAprep spin column in a collection tube, spun at 14,000 rpm for 60 seconds, the flow through was discarded and the column reassembled. Next, 750 μl of buffer PE was added and spun at 14,000 for 60 seconds , the flow through was discarded and the column reassembled. The column was spun at 14,000 rpm for 1 minute in order to dry it, and then the column was transferred to a new 1.5 ml tube. Finally, 50 μl of H₂O was added, incubated at room temperature for 1 minute and then spun at 14,000 rpm for 1 minute before discarding the column.

To transfect sf9 insect cells with Baculovirus DNA, 96.4 μl Excel Medium, 1 μl DiamondBac Cur Virus DNA (1 μg/μl), and 3.6 μl of the recombinant transfer plasmid in pVL1393 (1 μg) were assembled in a sterile 6 ml polystyrene tube. A mastermix of ESCORT and Excell 1:20 and add 100 μl to each polystyrene tube before incubating at room temp for 15 min to make the transfection mixture (475 μl Excell, 25 μl ESCORT). The monolayers of a 6 well plate of Sf9 cells were washed with a volume of 2 ml of Excell twice leaving the media on the cells after the second wash. After aspirating the wash media, 0.8 ml of Excell was added to each well of the 6 well plate before adding 0.2 ml of the transfection mixture to a well of the 6 well plate. This was incubated at 28° C. for 5 hours and then the transfection mixture was aspirated. The cells were washed once and then 2 ml of TNM-FH was added and incubated at 28° C. for 120-144 hours.

To harvest the transfections and fix cells for IFA, the supernatant from transfected Sf9 cells was aseptically harvested in a biosafety hood and transferred into a 2.0 ml cryovial before adding 1 ml of cold Acetone: Methanol (50:50) to the remaining Sf9 cells in the well. This was incubated at room temperature for 10 minutes, the fixative was removed and the plate was allowed to air dry in the fume hood before storing the plate at 4° C. or colder for eventual IFA.

To test for expression of M2ae1 in the Sf9 transfected cells, an indirect immunofluorescent assay (IFA) was performed. The plate was washed briefly in PBS to rehydrate the fixed cells and then the PBS was removed. Next, 500 μl of the primary antibody, αM2ae1, was added to the well and incubated for 1 hour at 37° C. The primary antibody was removed and the well washed three times with PBS with the final wash being removed. Next, 500 μl of the secondary antibody, a rabbit FITC 1:500 in PBS, was added to the well and incubated for 1 hour at 37° C. before removing the secondary antibody and washing the well three times with PBS and removing the final wash. Next, the well was coated with about 0.5 ml of Glycerol: Water (50:50) and the excess Glycerol: Water was removed so that the cell layer could be observed with an inverted UV light microscope.

To find a clone of baculovirus-infected Sf9 cells, a limiting dilution of baculovirus was performed on passage 50 of Sf9 cells. Briefly, 10 fold dilutions of baculovirus material into TNM-FH was performed. Just before performing the dilution, the baculovirus material was vortexed briefly to ensure it was mixed thoroughly. The initial 10⁻¹ dilution was performed by pipetting 0.1 ml of the baculovirus into 0.9 ml of TNM-FH and vortexing briefly to mix. The 10⁻² dilution was performed by pipetting 0.1 ml of the 10⁻¹ diluted baculovirus into 0.9 ml of TNM-FH and vortexing briefly to mix. The 10⁻³ dilution was performed by pipetting 1 ml of the 10² diluted baculovirus into 9 ml of TNM-FH and vortexing briefly to mix. The 10⁻⁴ and each subsequent dilution (up to 10⁻⁷) was performed by sequentially pipetting 1 ml of the 10⁻³ diluted baculovirus into 9 ml of TNM-FH and vortexing briefly to mix. The diluted baculovirus material was added to as many wells of the 96 well plate as possible. The plates were stacked and placed into a large zip-loc bag and incubated at 28° C. in the dark. Supernatants are then harvested from the wells after 4-7 days.

To fix the cells and stain the M2ae1 ORF2 plates for viewing under UV light, the supernatant was aseptically transferred into a new 96 well plate using a multi-channel pipettor and sterile filter tips in a biosafety hood. The supernatant-containing plates can be stored at 4° C. for short term storage or at −70° C. for long-term storage. The remaining Sf9 cells in the well had 200 μl of cold Acetone:Methanol (50:50) added thereto and this was incubated at room temperature for 10 minutes. The fixative was removed and the plate was air dried in the fume hood before washing the plate briefly in PBS to rehydrate the fixed cells. The PBS was then removed and 100 μl of Influenza A M2 (14C2) (Santa Cruz Biotech) was added to the well and incubated for 1 hour at 37° C. The primary antibody was then removed and the well was washed three times with PBS with the final wash being removed. Next, 100 μl of the secondary antibody, goat+mouse FITC, was added to the well and incubated for 1 hour at 37° C. The secondary antibody was then removed and the well was washed three times with PBS with the final wash being removed. The well was coated with about 0.5 ml of Glycerol:Water (50:50) before removing the excess Glycerol:Water and observing the cell layer with an inverted UV microscope.

A single M2ae1 ORF2 PCV2 baculovirus was isolated by limiting dilution of passage 1 on 96 well Sf9 plates, passage 52. Briefly, 10 fold dilutions of the baculovirus material into TNM-FH was performed. Just before performing the dilution, the baculovirus material was vortexed briefly to ensure it was mixed thoroughly. The initial 10⁻¹ dilution was performed by pipetting 0.1 ml of the baculovirus into 0.9 ml of TNM-FH and vortexing briefly to mix. The 10⁻² dilution was performed by pipetting 0.1 ml of the 10⁻¹ diluted baculovirus into 0.9 ml of TNM-FH and vortexing briefly to mix. The 10⁻³ dilution was performed by pipetting 0.1 ml of the 10⁻² diluted baculovirus into 9 ml of TNM-FH and vortexing briefly to mix. The 10⁻⁴ and each subsequent dilution (up to 10⁻⁶) was performed by sequentially pipetting 0.1 ml of the preceeding (e.g. 10⁻³ for the 10⁻⁴ dilution) diluted baculovirus into 0.9 ml of TNM-FH and vortexing briefly to mix. The 10⁻⁷ dilution was performed by pipetting 1 ml of the 10⁻⁶ diluted baculovirus into 9 ml of TNM-FH and vortexing briefly to mix. The 10⁻⁸ dilution was performed by pipetting 1 ml of the 10⁻⁷ diluted baculovirus into 9 ml of TNM-FH and vortexing briefly to mix. Next, 0.1 ml of the 10⁻⁷ and 10⁻⁸ diluted baculovirus material was added to as many wells of the 96 well plate as possible. The plates were stacked and placed into a large zip-loc bag and incubated at 28° C. in the dark. Supernatants are then harvested from the wells after 4-7 days.

To perform IFA on the 10⁻⁷ and 10⁻⁸ limiting dilutions, the Sf9 cells were fixed and stained to detect the presence of M2ae1 ORF2 PCV2 transfected cells. Briefly, the supernatant was aseptically transferred into a new 96 well plate using a multi-channel pipettor and sterile filter tips in a biosafety hood. The supernatant-containing plates can be stored at 4° C. for short term storage or at −70° C. for long-term storage. The remaining Sf9 cells in the well had 200 μl of cold Acetone: Methanol (50:50) added thereto and this was incubated at room temperature for 10 minutes. The fixative was removed and the plate was air dried in the fume hood before washing the plate briefly in PBS to rehydrate the fixed cells. The PBS was then removed and 100 μlof the primary antibody, Influenza A M2 (14C2) (Santa Cruz Biotech), was added to the well and incubated for 1 hour at 37° C. The primary antibody was then removed and the well was washed three times with PBS with the final wash being removed. Next, 100 μl of the secondary antibody, goat+mouse FITC, was added to the well and incubated for 1 hour at 37° C. The secondary antibody was then removed and the well was washed three times with PBS with the final wash being removed. The well was coated with about 0.5 ml of Glycerol:Water (50:50) before removing the excess Glycerol:Water and observing the cell layer with an inverted UV microscope. The results were positive.

To amplify the M2ae1 ORF2 PCV2 from the limiting dilutions above, supernatant was used. Briefly, the methodology added 100 μl of the respective virus to a single well of a 6 well plate of Sf9 cells before placing the plate at 28° C. in the dark. The supernatant could be harvested and cell layer fixed 4-7 days later.

Selected M2ae1 internal PCV2 ORF2 baculovirus were then subjected to limiting dilution amplification harvest and Sf9 cell fixation. Briefly, supernatant from transfected Sf9 cells was aseptically harvested in a biosafety hood and transferred into a 2.0 ml cryovial before adding 1 ml of cold Acetone: Methanol (50:50) to the remaining Sf9 cells in the well. This was incubated at room temperature for 10 minutes, the fixative was removed and the plate was allowed to air dry in the fume hood before storing the plate at 4° C. or colder for eventual IFA.

### Results

The presence of the M2ae1 fragment was confirmed and its immunogenicity or antigenicity detectable using the above-referenced methods.

### EXAMPLE 6

This example demonstrates that the Influenza A M2ae1 region can be inserted as an amino tail to the PCV2 ORF2 VLP.

M2ae1 24-mer with 3′-KpnI

The amino acid sequence of the M2ae1 24-mer is MSLLTEVETPIRNEWGCRCNDSSD (SEQ ID NO. 6). The M2ae1 24 amino acid sequence was reverse translated into nucleotide sequence using the optimal codon usage for Drosophila. Using specific oligonucleotide primers, PCR was performed to add a 3′-KpnI restriction enzyme site to the M2ae1 coding region.

Introduction of KpnI Restriction Site onto the 5′-End of the PCV2 ORF2 Coding Region

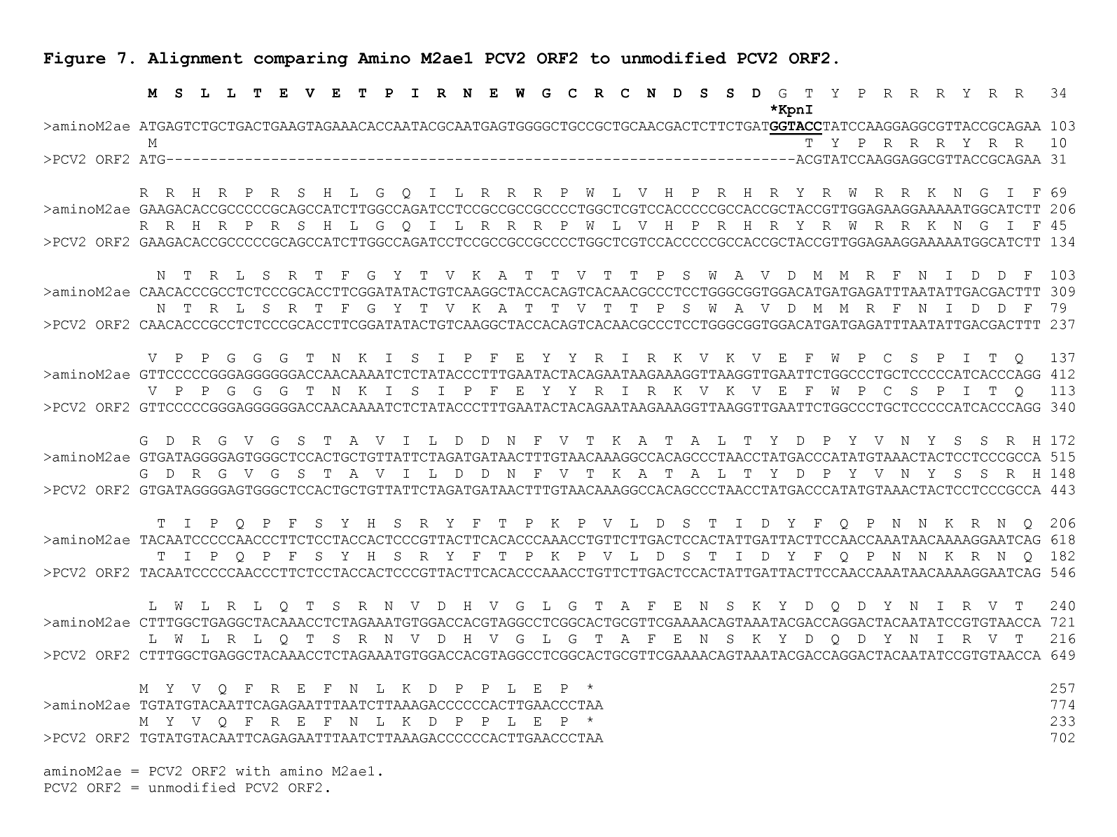

Using specific oligonucleotide primers, PCR was performed to add a KpnI restriction enzyme site to the 5′-end of the PCV2 ORF2 gene (refer to FIG. 7).

Joining of Amino M2ae1 onto the 5′-End of PCV2 ORF2

Using standard molecular biology methods, the M2ae1-KpnI region was cloned into the 5′-end KpnI site of the PCV2 ORF2 gene (SEQ ID NO. 12). The Amino M2ae1 PCV2 ORF2 region was then cloned into the baculovirus transfer vector, pVL1393. The resulting Amino M2ae1PCV2 ORF2/pVL1393 plasmid was then purified using the Qiagen Mini-Prep plasmid kit for subsequent use in transfection.

Generation of Recombinant Baculovirus Containing the PCV2 ORF2 with M2ae1 Tail

The Amino M2ae1PCV2 ORF2/pVL1393 plasmid and the DiamondBac® linearized baculovirus DNA (Sigma) were cotransfected into Sf9 insect cells using the ESCORT transfection reagent (Sigma) for 5 hours at 28° C. The transfection medium was removed and the transfected cells were then gently washed, replenished with media, and incubated at 27° C. Five days later, the cell supernatant containing the generated recombinant baculovirus was harvested and stored at 4° C. The remaining transfected Sf9 cells were fixed with acetone:methanol and used in immunofluorescence assay (IFA) with the anti-Influenza A M2 monoclonal antibody 14C2 to verify the expression of the M2ae1 region transfected Sf9 cells.

The harvested PCV2 ORF2 amino M2ae1 Baculovirus DB supernatant was used for generation of virus stock material.

### Immunological Detection of PCV2 ORF2 Amino M2ae1

Verification of M2ae1 expression in PCV2 ORF2 amino M2ae1 Baculovirus DB-infected Sf9 cells was previously confirmed by IFA. However, as a means to further confirm the expression of M2ae1 along with PCV2 ORF2, an immunoblot on PCV2 ORF2 amino M2ae1 harvested supernatant from baculovirus-infected insect cell cultures was performed.

Briefly, harvested supernatant from baculovirus-infected insect cell cultures was blotted onto PVDF membranes and the presence of PCV2 ORF2 and/or M2ae1 antigens were tested in an immunoblot. The primary antibodies used for immunoblot detection of PCV2 ORF2 were the anti-PCV2 ORF2 monoclonal antibody 6C4-2-4A3-5D10 and purified swine anti-PCV2 ORF2 IgG. The primary antibodies used for immunoblot detection of M2ae1 were the anti-M2 monoclonal antibody 14C2 (Santa Cruz Biotechnology, Inc.) and swine anti-M2aeC5 serum. The respective secondary antibodies used in the immunoblot were HRP-labeled goat anti-mouse conjugate and goat-anti-swine conjugate. Opti-4CN substrate (BioRad) was used for colorimetric detection on the immunoblots. The immunoblots revealed the presence of ORF2 and M2ae1.

1. An immunogenic composition comprising a PCV2 virus-like particle (VLP), wherein said VLP further comprises:
a. an amino acid segment from PCV2 consisting of the sequence of SEQ ID NO. 10; and
b. a foreign amino acid segment attached to said PCV2 amino acid segment at the amino or carboxyl terminus of said PCV2 amino acid segment, said foreign amino acid segment being from an organism other than PCV2, said foreign amino acid segment being from an organism other than PCV2.

2. (canceled)

3. (canceled)

4. (canceled)

5. The composition of claim 1, said foreign amino acid segment being detectable separately from said PCV2 amino acid segment.

6. The composition of claim 1, said foreign amino acid segment being detectable by an assay specific for said foreign amino acid segment.

7. The composition of claim 6, said assay comprising monoclonal antibodies.

8. (canceled)

9. The composition of claim 1, said composition inducing an immunological response in an animal receiving an administration thereof.

10. The composition of claim 9, said immunological response being specific to said foreign amino acid segment.

11. The composition of claim 9, said immunological response being sufficient to reduce the incidence of or lessen the severity of clinical, pathological, and/or histopathological signs of infection.

12. (canceled)

13. (canceled)

14. The composition of claim 1, said foreign amino acid segment being derived from an organism selected from the group consisting of Cryptosporidium parvum, swine influenza, and combinations thereof.

15. The composition of claim 14, said foreign amino acid segment having at least 80% sequence homology with a sequence selected from the group consisting of SEQ ID NOS. 1 and 6.

16. The composition of claim 14, said composition reducing the incidence of or severity of infection by Cryptosporidium parvum, swine influenza, and combinations thereof.

17. The composition of claim 1, said foreign amino acid segment having a length of about 8 to about 200 amino acids.

18. The composition of claim 1, further comprising an ingredient selected from the group consisting of adjuvants, pharmaceutical acceptable carriers, protectants, stabilizing agents, and combinations thereof.

19. An expression vector comprising:
a. vector DNA; and
DNA derived from PCV2, said DNA derived from PCV2 being from PCV2 ORF2; and
b. DNA derived from a second organism species, wherein said first and second species are different from one another and different from the organism species from which the vector DNA was derived, said DNA derived from a second organism species encodes a foreign amino acid segment attached to said PCV2 amino acid segment at the amino or carboxyl terminus of said PCV2 amino acid segment.

20. The expression vector of claim 19, said vector DNA being from a baculovirus.

21. (canceled)

22. (canceled)

23. The expression vector of claim 20, said PCV2 ORF2 DNA having the sequence of SEQ ID NO. 7.

24. The expression vector of claim 21, said DNA from a second organism species encoding an amino acid segment that induces an immunological response in an animal receiving an administration thereof.

25. The expression vector of claim 24, said immunological response reducing the incidence of or severity of clinical, pathological, or histopathological signs of infection from said first organism species, said second organism species, and combinations thereof.

26. The expression vector of claim 19, said DNA from a second species being selected from the group consisting of Cryptosporidium parvum, E. coli, and combinations thereof.

27. The expression vector of claim 26, said DNA from a second species encoding an amino acid segment selected from the group consisting of SEQ ID NOS. 1 and 6.

28. A method of producing antigen as a PCV2 virus-like particle (VLP) comprising the steps of:
a. combining DNA encoding said antigen with PCV2 DNA to produce a combined DNA insert wherein said PCV2 DNA encodes an amino acid segment from PCV2, said PCV2 amino acid segment consists of the sequence of SEQ ID NO:. 10; and said DNA encoding said antigen encodes a foreign amino acid segment attached to said PCV2 amino acid segment at the amino or carboxyl terminus of said PCV2 amino acid segment; and
b. expressing said combined DNA insert in an expression system.

29. The method of claim 28, said antigen having a length of about 8-200 amino acids.

30. The method of claim 28, said antigen being derived from an organism species different from PCV2.

31. The method of claim 30, said organism species being selected from the group consisting of Cryptosporidium parvum, swine influenza, and combinations thereof.

32. (canceled)

33. (canceled)

34. The method of claim 28, said ORF2 DNA having at with the sequence of SEQ ID NO. 7.

35. The method of claim 28, said expression system comprising a Baculovirus expression system.

36. The use of an antigen expressed by the method of claim 28 in a vaccine or an immunogenic composition.

37. The use of PCV2 ORF2 as a virus-like particle.


SEQUENCE LISTING (13 sequences)

SEQ ID NO: 1 - protein, 30 aa, Cryptosporidium parvum
AINGGGATLPQKLYLTPNVLTAGFAPYIGV

SEQ ID NO: 2 - DNA, 21 bp, Porcine circovirus
  misc_feature: Primer
tggatccgccatgacgtatcc

SEQ ID NO: 3 - DNA, 120 bp, Artificial Sequence
  Primer
agatctacacgccgatgtagggggcgaagccggcggtcagcacgttgggggtcaggtacagcttctggggcagggtggcgccgccgccgttgatggcgggttcaagtggggggtctttaa

SEQ ID NO: 4 - DNA, 806 bp, Artificial Sequence
  PCV2 ORF2 with cryptosporidium parvum ligand tail
tggatccgccatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaacccgccatcaacggcggcggcgccaccctgccccagaagctgtacctgacccccaacgtgctgaccgccggcttcgccccctacatcggcgtgtagatct

SEQ ID NO: 5 - protein, 266 aa, Artificial Sequence
  Translation of PCV2 ORF2 with CSL tail
GSAMTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEPAINGGGATLPQKLYLTPNVLTAGFAPYIGV

SEQ ID NO: 6 - protein, 24 aa, swine influenza virus
MSLLTEVETPIRNEWGCRCNDSSD

SEQ ID NO: 7 - DNA, 702 bp, Porcine circovirus
atgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaa

SEQ ID NO: 8 - DNA, 702 bp, Artificial Sequence
  PCV2 ORF2 with AscI restriction enzyme site
atgacgtatccaaggaggcgttaccgcagaagaagacaccgcccccgcagccatcttggccagatcctccgccgccgcccctggctcgtccacccccgccaccgctggcgcgcccgaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaa

SEQ ID NO: 9 - DNA, 783 bp, Artificial Sequence
  PCV2 ORF2 and swine influenza M2ae1
atgacgtatccaaggaggcgttaccgcagaagaagacaccgcccccgcagccatcttggccagatcctccgccgccgcccctggctcgtccacccccgccaccgctggcgcgccatgagcctgctgaccgaggtggagacccccatccgcaacgagtggggctgccgctgcaacgatagcagcgatcggcgcgcccgaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaa

SEQ ID NO: 10 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 11 - protein, 260 aa, Artificial Sequence
  PCV2 ORF2 and internal swine influenza M2ae1
MTYPRRRYRRRRHRPRSHLGQILRRRPWLVHPRHRWRAMSLLTEVETPIRNEWGCRCNDSSDRRARRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 12 - DNA, 774 bp, Artificial Sequence
  PCV2 ORF2 and amino swine influenza M2ae1
atgagtctgctgactgaagtagaaacaccaatacgcaatgagtggggctgccgctgcaacgactcttctgatggtacctatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcacaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccactattgattacttccaaccaaataacaaaaggaatcagctttggctgaggctacaaacctctagaaatgtggaccacgtaggcctcggcactgcgttcgaaaacagtaaatacgaccaggactacaatatccgtgtaaccatgtatgtacaattcagagaatttaatcttaaagaccccccacttgaaccctaa

SEQ ID NO: 13 - protein, 257 aa, Artificial Sequence
  PCV2 ORF2 and amino swine influenza M2ae1
MSLLTEVETPIRNEWGCRCNDSSDGTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP